(19) United States
(12) Patent Application Publication
(10) Pub. No.: US 2003/0153035 A1
(43) Pub. Date: Aug. 14, 2003

(54) Mutated bmp1b receptor as regulator of ovulation rate

(76) Inventors: Theresa Mary Wilson, Dunedin (NZ); Xi-Yang Wu, Sydney (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/169,051

(22) PCT Filed: Dec. 22, 2000
(86) PCT No.: PCT/NZ00/00259

(30) Foreign Application Priority Data
Dec. 23, 1999 (NZ) 502058

(51) Int. Cl. C07K 14/705; C07H 21/04; C12P 21/02; C12N 5/06

(52) U.S. Cl. 435/69.1; 435/320.1; 435/325; 530/350; 536/23.5

(57) ABSTRACT

The present invention is related to an isolated mutated nucleic acid molecule encoding the BMP1B receptor polypeptide. This molecule has a sequence which differs from that of the wild type BMP1B receptor polypeptide in that the codon encoding amino-acid residue 249 encodes arginine rather than glutamine, is able to hybridize under stringent conditions to the molecule above, is a variant of the molecule above, is a complement of any of the molecules above, or is an anti-sense sequence corresponding to any of the sequences in the molecules described above.

The present invention concerns variation of ovulation rate in animals. In one aspect a mutation in a gene is involved in increasing the ovulation rate in heterozygous and homozygous female vertebrates. The mutated gene sequence can be used in a test to identify heterozygous or homozygous female and male vertebrates carrying said mutated gene. In a further aspect the invention relates to identification of the protein responsible for determining the ovulation rate in vertebrates. In yet a further aspect the invention concerns modulation of the activity of this protein to control the ovulation rate in female vertebrates.

### BACKGROUND OF THE INVENTION

It will be clearly understood that, although a number of prior art publications are referred to herein, this reference does not constitute an admission that any of these documents form part of the common general knowledge in the art, in New Zealand or in any other country.

The Booroola Merino rates among the top breeds of sheep in the world in terms of ovulation rate. Sheep derived from the Booroola Merino strain carry a major autosomal mutation that increases ovulation and litter size (Davis et al 1982), and the mutation has been named FecB (fecundity). The effect of FecB is additive for ovulation rate (ovulation rate increasing by about 1.5 for each copy) and on average, one copy of FecB increases litter size by about one extra lamb and two copies increase litter size by about 1.5 lambs. Homozygotes FecB^{B}/FecB^{B} (BB), heterozygotes FecB^{B}/FecB⁺ (B+) and noncarriers FecB⁺/FecB⁺ (++) of the Booroola gene can be segregated on the basis of ovulation-rate recordings. The physiological effects of the FecB gene have been extensively characterised (McNatty et al 1986, 1987, Hudson et al 1999). There is evidence that the high ovulation rate of the FecB^{B} FecB^{B} ewes may be related to an alteration in intraovarian regulation (Fry et al 1988, McNatty et al 1993)

Application of the Booroola Gene in the Sheep Industry

A Booroola ram is currently of added value if the carrier status of the ram is known. Rams carrying the Booroola gene have been exported to many countries, including France, Britain, South Africa, Poland, Chile, Israel, Netherlands and the USA, with the intention of introgressing the high lambing found in the Booroola into their own flocks.

Test for Inheritance

The FecB mutation in sheep is linked to markers from a region of syntenic homology to human chromosome 4q21-25, and has been mapped to sheep chromosome 6q23-31 (Montgomery et al_{—}1994). The linkage to known markers can identify the Booroola gene carrier status of sheep. A commercial test provided by Genomnz, a commercial unit within AgResearch, New Zealand is based on the inheritance of a chromosome region defined by polymorphic microsatellite markers. The Booroola genotype can only be assigned when at least one animal has a known relationship between the chromosomal region and FecB so the test is limited to clients who have FecB, segregating within their flocks, and for whom samples are available from confirmed FecB carriers. Another problem with the test is that the Booroola test markers are genetically far enough apart for crossovers to occur between the markers. Whenever this occurs, it is not possible to assign the Booroola status of an animal, and this is expected to occur in approximately 10% of samples.

Transforming Growth Factor Beta Family

The proteins of the transforming growth factory-β (TGF-β) super-family, which includes TGF-βs and bone morphogenetic proteins (BMP's), are multifunctional proteins that regulate growth, differentiation and extracellular matrix production in many cell types (Helden et al 1997, Massague 1998). Members of this family play essential roles during embryogenesis in mammalians, amphibians and insects as well as in bone development. The mechanism whereby TGF-β and related factors mediate their biological effects is of great interest. Recent work has elucidated how several members of this family initiate signalling from the cell surface. They exert their cellular actions through distinct complexes of type I and type II serine/threonine kinases. Both receptor types are essential for signalling; the type I receptor acts downstream of the type II receptor and determines signal specificity. Upon binding the type II receptor, phosphorylates the type I receptor and activates this kinase. In turn, the activated type I receptor propagates the signal to downstream substrates, using the Smad proteins as carriers of the signal (Kretzschmar et al 1997).

BMP1B Receptor

BMP1B receptor is a member of the transforming growth factors family and interacts with the Smad proteins, which have pivotal roles in the intracellular signal transduction of the TGF-β family members. The existence of a functional BMP system in the ovary has been established. The family of BMP receptors, BMPR-IA, -IB and -II are expressed in a cell-type specific manner in the normal cycling rat ovary, with high levels of expression found in the granulosa cells surrounding the dominant follicle (Shimasaki et al 1999)

The applicant has found that a mutation in the sheep BMP1B receptor gene is responsible for the increased ovulation rate seen in sheep carrying the Booroola gene.

The role of the BMP1B receptor in fecundity was previously unknown.

### SUMMARY OF THE INVENTION

Accordingly, in one aspect, the present invention provides an isolated mutated nucleic acid molecule encoding Bone Morphogenetic Protein IB (BMP1B) receptor wherein the molecule has a sequence differing from the wild type in that the codon encoding amino acid residue 249 encodes arginine not glutamine (hereinafter referred to as a mutated BMP1B receptor sequence), or the sequence is a biologically functional equivalent of the mutated sequence.

It will be clearly understood that the invention also encompasses nucleic acid molecules of sequences such that they are able to hybridize under stringent conditions to the mutated BMP1B receptor sequence, or which have greater than 80% sequence identity to this mutated sequence, with the proviso that this aspect of the invention excludes the wild type BMP1B receptor sequence. The invention also encompasses the complement of a nucleic acid molecule as defined above.

The nucleic acid molecule may be an RNA, cRNA, genomic DNA or cDNA molecule.

The present invention further provides a method for identifying a vertebrate which carries a mutated BMP1B receptor nucleic acid molecule, said method comprising the steps of:

i) obtaining a tissue or blood sample from the verterbrate;

ii) isolating DNA from the sample;

iii) optionally isolating BMP1B receptor DNA from DNA obtained at step ii);

iv) optionally probing said DNA with a probe complementary to the mutated BMP1B receptor molecule of claim 1, thereby to identify mutated BMP1B receptor;

v) optionally amplifying the amount of mutated BMP1B receptor DNA and;

vi) determining whether the mammal BMP1B receptor sequence DNZ obtained in step (ii) carries a mutation which is associated with increased or decreased ovulation rates.

Preferably the mutation is in the intracellular signalling domain of the BMP1B receptor DNA, more preferably within the codon encoding the amino acid corresponding to amino acid residue 249 in the sequence of FIG. 3a or SEQ ID No. 2.

The amplification in step (v) may be performed by any convenient method, such as polymerase chain reaction (PCR).

The vertebrates to which the present invention has application may be male or female, and may be a human; or a domestic, companion or zoo or feral mammal; or other warm blooded vertebrates.

The test may generally be used to assess fecundity in vertebrates such as humans and other commercially important mammals, and birds including sheep, cattle, horses, goats, deer, pigs, cats, dogs, possums, and poultry.

According to still a further aspect, the present invention provides a genetic marker useful for identifying vertebrates which have an enhanced rate of ovulation. The marker comprises a nucleic acid molecule which hybridises to a nucleotide sequence which encodes a BMP1B receptor sequence. Preferably the marker is able to specifically hybridize to:

a) the Booroola BMP1B DNA sequence of FIG. 2 wherein arginine is substituted for glutamine at amino acid residue 249, or the sequence set forth in SEQ ID No. 3; or variants thereof

b) a genomic DNA within or associated with the mutated BMP1B receptor gene, or a variant thereof, or

c) a complement any sequence to the sequences of a) and b).

Preferably the vertebrate is a human or one of commercial significance; more preferably the vertebrate is selected from the group consisting of sheep, cattle, horses, goats, deer, pigs, cats, dogs, mice, rats and poultry.

Preferably the genetic marker comprises the Booroola DNA sequence of:

a) FIG. 2 in which the bold A is substituted with a G; or

b) SEQ ID No. 3; or

c) A complement any sequence to the sequences of a) or b).

Most preferably, the genetic marker comprises at least Booroola DNA sequence of:

a) FIG. 3c or

b) SEQ ID No. 3 in the region which includes the codon encoding amino acid residue 243; or

c) Complement any sequence to the sequences of a) or b).

According to a still further aspect, the present invention provides a method of identifying vertebrates which have an enhanced ovulation rate, said method comprising the measurement in female vertebrates of the levels of a mutated BMP1B receptor polypeptide associated with vertebrates which have higher ovulation rates.

In a further aspect, the present invention provides a mutated BMP1B receptor polypeptide differing from the wild type in that residue 249 is arginine not glutamine; or a functional variant thereof which has the ability to modulate ovulation in a female vertebrate.

In a further aspect, the present invention provides an isolated polypeptide selected from the amino acid sequence of:

a) FIG. 3a; or

b) SEQ ID No. 2; or

c) a variant to the sequences of a) or b) which has the ability to modulate ovulation in a female mammal.

In a further aspect, the present invention a method of modulating the ovulation rate of a female vertebrate, said method comprising administering to said vertebrate an effective amount of an inhibitor or agonist of the BMP1B receptor.

A preferred method of this aspect uses a BMP1B receptor antibody. It will be clearly understood that for the purposes of this method the term “antibody” encompasses fragments or analogues of antibodies which retain the ability to bind to the BMP1B receptor, including but not limited to Fv, F(ab), and F(ab)₂ fragments, scFv molecules and the like. Preferably the antibody is a monoclonal antibody.

In yet a further aspect, the invention provides for the use of a composition comprising an effective amount of an inhibitor or agonist of the BMP1B receptor together with a pharmaceutically or veterinarily acceptable carrier. Preferably, for the use of a composition comprising an effective amount of agent selected from the group consisting of:

a) wild-type or mutated BMP1B receptor polypeptides, or an immunogenic region thereof;

(b) an antibody directed against wild-type or mutated BMP1B receptor polypeptide, or an antigen-binding fragment thereof;

(c) an antisense nucleic acid directed against nucleic acid encoding the mutated or wild-type BMP1B receptor polypeptide;

(d) a pseudoreceptor to the wild-type or mutated BMP1B receptor; and

(e) a ligand which binds to the wild-type or mutated BMP1B receptor polypeptide, to thereby inhibit the activity of the endogenous BMP1B receptor of the vertebrate;

and a pharmaceutically or veterinarily acceptable carrier, to modulate the ovulation rate of a vertebrate.

The term “pseudoreceptor” refers to a small protein or peptide which is based on the sequence of the BMP1B receptor and is capable of blinding an active ligand in a similar way to the endogenous BMP1B so as to modulate the ovulation rate.

In yet a further aspect, the invention provides a kit for identifying homozygous and/or heterozygous male and female vertebrates carrying the mutated BMP1B receptor gene by identifying either the nucleic acid sequences per se or the expressed protein of the mutated BMP1B gene.

While the invention is broadly as defined above, it will be appreciated by those persons skilled in the art that it is not limited thereto and that it also includes embodiments of which the following description gives examples.

### BRIEF DESCRIPTION OF THE DRAWINGS

Preferred aspects of the invention will be described in relation to the accompanying drawings in which:

FIG. 1 shows a Genetic linkage map of sheep chromosome 6. Genetic distances are in Kosamabi centiMorgans (cM). The Booroola (FecB) gene maps into the region indicated by the solid bar.

FIG. 1(B) Quantitative Trait Loci (QTL) analysis of the distribution of the test statistic (F-ratio) along chromosome 6 for the trait analysed. Ovulation rate was measured in early and late April at 2.5 years of age and at equivalent times at 3.5 years of age. These four traits were combined and the mean residual deviation from the population mean over all four tits was used in the analysis. Positions of markers are indicated along the x-axis.

FIG. 2 shows the nucleotide sequence of the BMP1B receptor in wild-type sheep. The position of the nucleotide substitution in Booroola sheep is the A at position 830 marked in bold. In Booroola sheep this nucleotide is G. The start codon (ATG) and the stop codon (TGA) are underlined.

FIG. 3a shows the deduced amino acid sequence of the BMP1B receptor polypeptide in wild-type sheep as encoded by the nucleotide sequence of FIG. 2. The amino acid at position 249 which is affected by the Booroola base substitution as position 249 is marked in bold.

FIG. 3b shows the wild-type sequence around amino acid residue 249.

FIG. 3c shows the Booroola sequence around amino acid residue 249.

FIG. 4 shows the high homology between sequences for BMP1B receptor gene in the species sheep, human, mouse and chick and the position of the mutation that is found in Booroola animals.

FIG. 5 shows the expression of the BMP1B receptor in different tissues of the sheep including the ovary.

FIG. 6 shows an example of a type of test that can be used to screen for the mutation. This test is called Forced RFLP and generates a restriction site for the enzyme AvaII in animals carrying the Booroola mutation.

### DETAILED DESCRIPTION OF THE INVENTION

We have shown for the first time that the mutations in the BMP1B receptor gene are responsible for the increased ovulation rates seen in animals heterozygous or homozygous for the Booroola gene.

Accordingly, in one aspect, the present invention provides an isolated mutated nucleic acid molecule encoding the BMP1B receptor polypeptide wherein the molecule

(a) has a sequence which differs from that of the wild type BMP1B receptor polypeptide in that the codon encoding amino-acid residue 249 encodes arginine or lysine rather than glutamine;

(b) is a non-wildtype variant of the sequence defined in (a) having an affect on modulation of ovulation;

(c) is the complement of the molecule defined in (a) or (b); or

(d) anti-sense sequences corresponding to any of the sequences in (a)-(c).

The nucleic acid molecule may be an RNA, cRNA, genomic DNA or cDNA molecule.

The term “isolated” means substantially separated or purified. from contaminating sequences in the cell or organism in which the nucleic acid naturally occurs and includes nucleic acids purified by standard purification techniques as well as nucleic acids prepared by recombinant technology, including PCR technology, and nucleic acids which have been synthesised. Preferably, the nucleic acid molecule is isolated from the genomic DNA of sheep expressing the Booroola phenotype.

The term “modulation of ovulation” means increasing or decreasing the rate of ovulation compared to the endogenous rate observed in a manual.

According to a further aspect, the present invention relates to a method for identifying a vertebrate which carries a mutated BMP1B receptor nucleic acid molecule, said method comprising the steps of:

i) obtaining a tissue or blood sample from the vertebrate;

ii) isolating DNA from the sample;

iii) optionally isolating BMP1B receptor DNA from DNA obtained at step ii);

iv) optionally probing said DNA with a probe complementary to the mutated BMP1B receptor molecule of claim 1, thereby to identify mutated BMP1B receptor;

v) optionally amplifying the amount of mutated BMP1B receptor DNA and;

vii) determining whether the mammal BMP1B receptor sequence DNA obtained in step (ii) carries a mutation which is associated with increased or decreased ovulation rates.

The probe and primers that can be used in this method also forms a part of this invention. Said probes and primers may comprise a fragment of the nucleic acid molecule of the invention capable of hybridising under stringent conditions to a mutated BMP1B receptor gene sequence. Such probes and primers are also useful, in studying the structure and function of the mutated gene, and for obtaining homologues of the gene from mammals other than sheep expressing the Booroola phenotype.

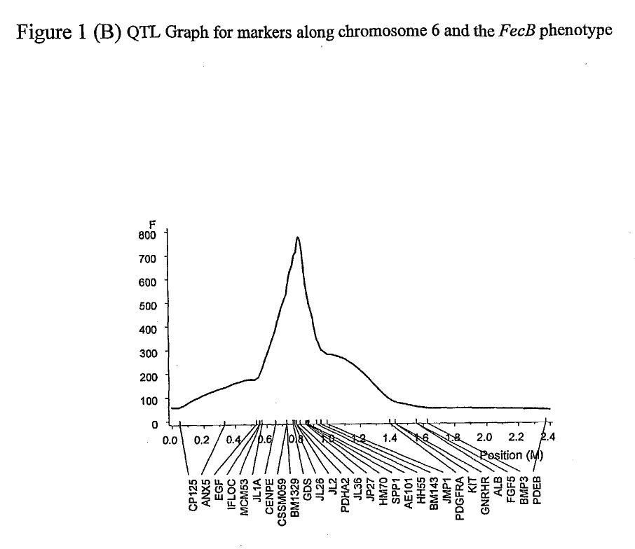

Nucleic acid probes and primers can be prepared based on nucleic acids according to the present invention eg the sequence of FIG. 2 with the bold A substituted by G or the sequence set forth in SEQ ID No. 3; or sequences complementary to these sequences. A “probe” comprises an isolated nucleic acid attached to a detectable label or reporter molecule. Typical labels include radioactive isotopes, ligands, chemiluminescent agents, and enzymes.

A “fragment” is a portion of the nucleic acid that is less than fall length and comprises at least a minimum sequence capable of hybridising specifically with a nucleic acid molecule according to the present invention or a sequence complementary thereto under stringent conditions as defined below. A fragment according to the invention has at least one of the biological activities of the nucleic acid or polypeptide of the invention.

“Primers” are short nucleic acids, preferably DNA oligonucleotides 15 nucleotides or more in length, which are annealed to a complementary target DNA strand by nucleic acid hybridization to form a hybrid between the primer and the target DNA strand, then extended along the target DNA strand by a polymerase, preferably a DNA polymerase. Primer pairs can be used for amplification of a nucleic acid sequence, eg by the polymerase chain reaction (PCR) or other nucleic acid amplification methods well known in the art. PCR-primer pairs can be derived from the sequence of a nucleic acid according to the present invention, for example, by using computer programs intended for that purpose such as Primer (Version 0.5© 1991, Whitehead Institute for Biomedical Research, Cambridge, Mass.).

Methods for preparing and using probes and primers are described, for example, in Sambrook et al. Molecular Cloning: A Laboratory Manual, 2nd ed, vol. 1-3, ed Sambrook et al. Cold Spring Harbour Laboratory Press, Cold Spring Harbour, N.Y., 1989.

Probes or primers can be free in solution or covalently or noncovalently attached to a solid support by standard means.

For the amplification of a target nucleic acid sequence (eg by PCR) using a particular amplification primer pair, stringent conditions are conditions that permit the primer pair to hybridise only to the target nucleic acid sequence to which a primer having the corresponding wild type sequence (or its complement) would bind.

Nucleic acid hybridization is affected by such conditions as salt concentration, temperature, or organic solvents, in addition to the base composition, length of the complementary strands, and the number of nucleotide base mismatches between the hybridising nucleic acids, as will be readily appreciated by those skilled in the art.

When referring to a probe or primer, the term “specific for (a target sequence)” indicates that the probe or primer hybridises under stringent conditions only to the target sequence in a given sample comprising the target sequence.

In another embodiment, the invention provides a genetic marker for increased ovulation rate in humans and other vertebrates such as sheep, goats, cattle, deer and pigs, or any other commercially important vertebrate. The invention provides a means of using a nucleic acid molecule containing sequence derived from a mutated BMP1B receptor DNA sequence, or genomic DNA that is associated with the mutated BMP1B receptor gene, to identify sequence variants in individual animals that are associated with increased ovulation of that animal. Although these variants may not necessarily give rise to the increased ovulation or sterility trait directly, they will be sufficiently closely associated with it to predict the trait. The methods by which these sequence variants are identified are known in the art, and include, but are not limited to, restriction fragment length polymorphism (RFLP), amplified fragment length polymorphism AFLP, direct sequencing of DNA within or associated with the mutated BMP1B receptor gene, or identification and characterisation of variable number of tandem repeats (VNTR), also known as microsatellite polymorphisms. Thus, the genetic marker may have utility in DNA selection of animals having increased ovulation.

The genetic marker may comprise at least one of the DNA sequences selected from the sequence of FIG. 2 in which the bold A is substituted by G or the sequence set forth in SEQ ID No. 3.

In a further aspect, the present invention provides a mutated BMP1B receptor polypeptide differing from the wild type in that residue 249 is arginine not glutamine; or a functional variant thereof which has the ability to modulate ovulation in a female mammal.

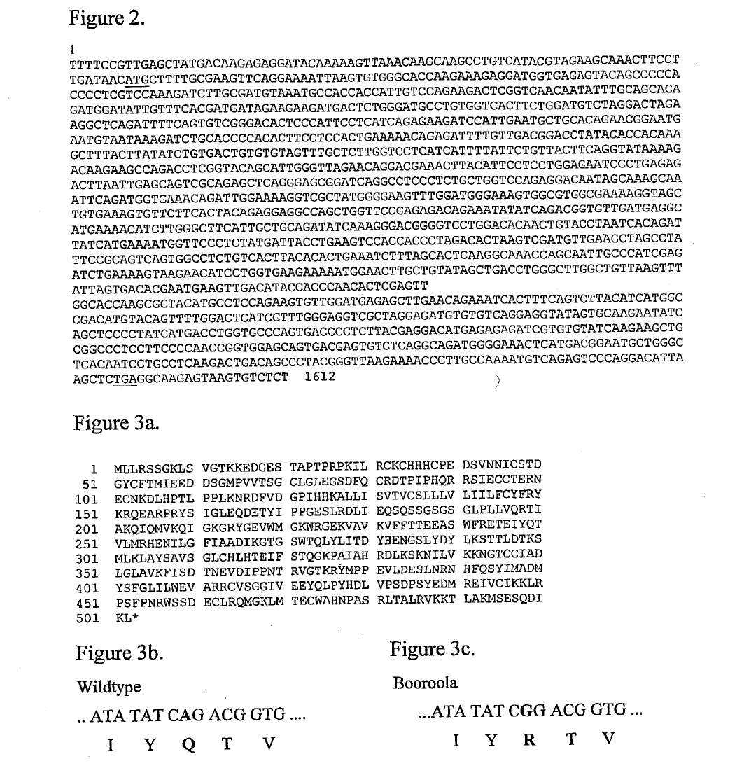

In a further aspect, the present invention provides an isolated polypeptide selected from the amino acid sequences of FIG. 3a or SEQ ID No. 2, or variants of these sequences which have the ability to modulate manipulate ovulation in a female mammal.

The polypeptide may be produced by expression of a suitable vector comprising the nucleic acid molecule encoding:

a) the polypeptide of FIG. 3a or SEQ ID No. 2 or variants of these sequences, or

b) the polypeptide of FIG. 3a with the arginine amino acid substitution at residue 249 (exemplified in FIG. 3c and SEQ ID No. 4); or variants of these sequences.

in a suitable host cell as would be understood by a person skilled in the art. The polypeptide may be incorporated into a pharmaceutically or veterinarily acceptable carrier such as isotonic saline for administration to a human or an animal for modulation of ovulation. The polypeptide may also be used to raise antibodies for use in other aspects of the invention.

The cloning vector may be selected according to the host or host cell to be used. Useful vectors will generally have the following characteristics:

(a) the ability to self-replicate;

(b) the possession of a single target for any particular restriction endonuclease; and

(c) desirably, carry genes for a readily selectable marker such as antibiotic resistance.

Two major types of vector possessing these characteristics are plasmids and bacterial viruses (bacteriophages or phages). Presently preferred vectors include plasmids pMOS-Blue, pGem-T, pUC8 and pcDNA3.

The DNA molecules of the invention may be expressed by placing them in operable linkage with suitable control sequences in a replicable expression vector. Control sequences may include origins of replication, a promoter, enhancer and transcriptional terminator sequences amongst others. The selection of the control sequence to be included in the expression vector is dependent on the type of host or host cell intended to be used for expressing the DNA.

Generally, procaryotic, yeast or mammalian cells are useful hosts. Also included within the term hosts are plasmid vectors. Suitable procaryotic hosts include E. coli, Bacillus species and various species of Pseudomonas. Commonly used promoters such as ∃β-lactamase (penicillinase) and lactose (lac) promoter systems are all well known in the art. Any available promoter system compatible with the host of choice can be used. Vectors used in yeast are also available and well known. A suitable example is the 2 micron origin of replication plasmid.

Similarly, vectors for use in mammalian cells are also well known. Such vectors include well known derivatives of SV-40, adenovirus, retrovirus-derived DNA sequences, Herpes simplex viruses, and vectors derived from a combination of plasmid and phage DNA.

Further eucaryotic expression vectors are known in the art (e.g. P. J. Southern and P. Berg, J. Mol. Appl. Genet. 1 327-341 (1982); S. Subramani et al.,Mol. Cell. Biol. 1, 854-864 (1981); R J. Kaufmann and P. A. Sharp, “Amplification and Expression of Sequences Cotransfected with a Modular Dihydrofolate Reducase Complementary DNA Gene, J. Mol. Biol. 159, 601-621 (1982); R J. Kaufmann and P. A. Sharp, Mol. Cell. Biol. 159, 601-664(1982); S. I. Scahill et al., “Expressions And Characterization Of The Product Of A Human Immune Interferon DNA Gene In Chinese Hamster Ovary Cells,” Proc. Natl. Acad. Sci. USA. 80, 4654-4659 (1983); G. Urlaub and L. A. Chasin, Proc. Natl. Acad. Sci. USA. 77, 4216-4220, (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the glycolytic promoters of yeast acid phosphatase, e.g. Pho5, the promoters of the yeast alpha-mating factors, and promoters derived from polyoma, adenovirus, retrovirus, and simian virus, e.g. the early and late promoters of SV40, and other sequences known to control the expression of genes of prokaryotic and eucaryotic cells and their viruses or combinations thereof.

In the construction of a vector it is also an advantage to be able to distinguish the vector incorporating the foreign DNA from unmodified vectors by a convenient and rapid assay. Reporter systems useful in such assays include reporter genes, and other detectable labels which produce measurable colour changes, antibiotic resistance and the like. In one preferred vector, the β-galactosidase reporter gene is used, which gene is detectable by clones exhibiting a blue phenotype on X-gal plates. This facilitates selection. In one embodiment, the β-galactosidase gene may be replaced by a polyhedrin-encoding gene; which gene is detectable by clones exhibiting a white phenotype when stained with X-gal. This blue-white color selection can serve as a useful marker for detecting recombinant vectors.

Once selected, the vectors may be isolated from the culture using routine procedures such as freeze-thaw extraction followed by purification.

For expression, vectors containing the DNA of the invention and control signals are inserted or transformed into a host or host cell. Some useful expression host cells include well-known prokaryotic and eucaryotic cells. Some suitable prokaryotic hosts include, for example, E. coli, such as E. coli, S G-936, E. coli HB 101, E. coli W3110, E. coli X1776, E. coli, X2282, E. coli, DHT, and E. coli, MR01, Pseudomonas, Bacillus, such as Bacillus subtilis, and Streptomyces. Suitable eucaryotic cells include yeast and other fungi, insect, animal cells, such as COS cells and CHO cells, human cells and plant cells in tissue culture.

Depending on the host used, transformation is performed according to standard techniques appropriate to such cells. For prokaryotes or other cells that contain substantial cell walls, the calcium treatment process (Cohen, S N Proceedings, National Academy of Science, USA 69 2110 (1972)) may be employed. For mammalian cells without such cell walls the calcium phosphate precipitation method of Graeme and Van Der Eb, Virology 52:546 (1978) is preferred. Transformations into plants may be carried out using Agrobacterium tumefaciens (Shaw et al., Gene 23:315 (1983) or into yeast according to the method of Van Solingen et al. J. Bact. 130: 946 (1977) and Hsiao et al. Proceedings, National Academy of Science, 76: 3829 (1979).

Upon transformation of the selected host with an appropriate vector the polypeptide or peptide encoded can be produced, often in the form of fusion protein, by culturing the host cells. The polypeptide or peptide of the invention may be detected by rapid assays as indicated above. The polypeptide or peptide is then recovered and purified as necessary. Recovery and purification can be achieved using any of those procedures known in the art, for example by absorption onto and elution from an anion exchange resin. This method of producing a polypeptide or peptide of the invention constitutes a further aspect of the present invention.

Host cells transformed with the vectors of the invention also form a further aspect of the present invention.

The term “variant” as used herein refers to nucleotide and polypeptide sequences wherein the nucleotide or amino acid sequence exhibits substantially 60% or greater homology with the nucleotide or amino acid sequence of the Figures, preferably 75% homology and most preferably 90-95% homology to the sequences of the present invention.—as assessed by GAP or BESTFIT (nucleotides and peptides), or BLASTP (peptides) or BLAST X (nucleotides). The variant may result from modification of the native nucleotide or amino acid sequence by such modifications as insertion, substitution or deletion of one or more nucleotides or amino acids or it may be a naturally-occurring variant. The term “variant” also includes homologous sequences which hybridise to the sequences of the invention under standard hybridisation conditions defined as 2×SSC at 65° C., or preferably under stringent hybridisation conditions defined as 6×SCC at 55° C., provided that the variant is capable modulating the ovulation rate of a female mammal. Where such a variant is desired, the nucleotide sequence of the native DNA is altered appropriately. This alteration can be effected by synthesis of the DNA or by modification of the native DNA, for example, by site-specific or cassette mutagenesis. Preferably, where portions of cDNA or genomic DNA require sequence modifications, site-specific primer directed mutagenesis is employed, using techniques standard in the art.

The term “protein (or polypeptide)” refers to a protein encoded by the nucleic acid molecule of the invention, including fragments, mutations and homologues having the same biological activity, ie the ability to modulate the ovulation rate. The polypeptide of the invention can be isolated from a natural source, produced by the expression of a recombinant nucleic acid molecule, or can be chemically synthesised.

In addition, nucleotides and peptides having substantial identity to the nucleotide and amino acid sequences of the invention can also be employed in preferred embodiments. Here “substantial identity” means that two sequences, when optimally aligned such as by the programs GAP or BESTFIT (nucleotides and peptides) using default gap weights, or as measured by computer algorithm BLASTP (peptides) and BLASTX (nucleotides), share at least 60%, preferably 75%, and most preferably 90-95% sequence identity. Preferably, residue positions which are not identical differ by conservative amino acid substitutions. For example, the substitution of amino acids having similar chemical properties such as charge or polarity is, not likely to affect the properties of a protein. Examples of such substitution include glutamine for asparagine or glutamic acid for aspartic acid.

In other aspects, the invention provides a method of reducing the ovulation rate in a female vertebrate comprising the step of inducing an immune response to mutated or wild-type BMP1B receptor polypeptide. This may represent either active or passive immunity. Alternatively antisense nucleic acid, a pseudoreceptor or an inhibitory ligand may be used.

Thus the method provides a method of reducing the ovulation of a female mammal comprising the step of administering an effective amount of an agent selected from the group consisting of:

(a) an immunising-effective amount of wild-type or mutated BMP1B receptor polypeptide, or an immunogenic region thereof,

(b) an antibody directed against wild-type or mutated BMP1B receptor polypeptide, or an antigen-binding fragment thereof;

(c) an antisense nucleic acid directed against nucleic acid encoding the mutated or wild-type BMP1B receptor polypeptide;

(d) a pseudoreceptor to the wild-type or mutated BMP1B receptor; and

(e) a ligand which binds to the wild-type or mutated BMP1B receptor polypeptide, to thereby inhibit the activity of the endogenous BMP1B receptor of the vertebrate.

And a pharmaceutically or veterinarily acceptable carrier and wherein said composition modulates the ovulation rate.

Alternatively, antisense nucleic acid for example stable antisense RNA may be used to manipulate the BMP1B receptor activity and consequently the ovulation rate. This may be carried out by a method analogous to that used by Hussainus et at (1999) for neutralising the activity of LDL receptor-related protein.

A further alternative is the use of a pseudo receptor analogous to that described by Onichtchouck et al (1999) for silencing of TGF-beta signalling,

An additional aspect of the present invention provides a ligand that binds to a polypeptide of the invention, and inhibits its activity. Most usually, the ligand is an antibody or antigen binding fragment thereof. Such ligands also form a part of this invention.

It will be appreciated that the reduction in ovulation rate may be sufficiently complete and/or long lasting to constitute sterilization of the vertebrate.

Thus, the present invention may have utility in reducing unwanted populations of feral vertebrates.

In a further aspect, the invention provides a method of producing an antibody to said polypeptide of the invention, comprising the steps of:

(a) expressing a suitable vector comprising the nucleic acid molecule of the invention or functional variant thereof, in a suitable host cell;

(b) recovering the expressed polypeptide or peptide; and

(c) raising monoclonal or polyclonal antibodies to said polypeptide or peptide by methods known in the art.

According to a further aspect, there is provided a composition comprising a polypeptide or nucleic acid of the invention and a pharmaceutically or veterinarily acceptable carrier such as would be known to a person skilled in the art. More than one polypeptide or nucleic acid of the invention can of course, be included in the composition. The carrier may be an isotonic saline solution.

According to a still further aspect of the present invention there is provided a kit for identifying male and female vertebrates which carry a single (heterozygous) copy and females carrying two (homozygous) copies of a mutated BMP1B receptor nucleic acid molecule of the invention, said kit comprising:

| | |
|---|---|
| X | primer pairs for amplification of the appropriate region of BMP1B |
| | receptor gene; and optionally one or more of |
| X | buffered salt solution for the amplification, such as PCR |
| | amplification; |
| X | deoxynucleotide mixtures; |
| X | thermostable DNA polymerase enzyme; |
| X | control DNA from the species being tested; |
| X | appropriate standards; |
| X | an appropriate detection system which could comprise one of the |
| | primers in each pair being labelled fluorescently or otherwise, or a |
| | labelled probe for detection of the product; and |
| X | instructions and protocols for the amplification, and subsequent |
| | detection of the amplification products and interpretation of results. |

The invention also provides a kit for detecting circulating mutated BMP1B receptor protein in a vertebrate comprising a specific antibody to the mutated BMP1B receptor protein. Such a kit may comprise a standard ELISA or enzyme immunoassay format kit familiar to those skilled in the art, for example it could comprise the antibody, and standard secondary antibody amplification components to enhance the signal. The antibodies may be conjugated to a fluorescent or radioactive or chemiluminescent label, or the secondary antibody may be labelled. Appropriate solutions, controls, buffers, instructions and protocols may optionally also be supplied.

Non-limiting examples of the invention will now be provided.

### EXAMPLE

Animals

The animals used in the mapping study were from the AgResearch Booroola half-sib and backcross flock. Fifteen B+ rams were mated with ++ ewes and generated 540 half-sib daughters. For the backcross families, BB rams were mated with ++ ewes and their B+ daughters mated with ++ rams to follow the inheritance through 3-4 generations (249 animals in total). Female progeny were measured by laparoscopy twice at consecutive cycles at approximately 19 and 31 months of age to identify animals carrying the Booroola phenotype and also by analysis of the inheritance of the microsatellite markers that map close to the FecB gene.

Mapping

For initial mapping studies, markers from chromosome 6 were typed in DNA samples from the half-sib and backcross flocks. FecB genotypes were assigned on the basis of records of ovulation rate as previously described [Montgomery et al, 1994], except that an additional constraint was placed on the half-sib family members before a genotype was assigned. This constraint required that the mean ovulation rate was not in the central 10% of mean ovulation rates for that family, and was used to account for the differences in mean ovulation rate across families. The FecB genotype was mapped onto the OOV6 map using the ‘all’ option of CRI-MAP, as previously described [Crawford et al, 1995] to find the intervals with lod 3 support.

DNA Purification and Sequencing

DNA was purified from the white blood cells present in 5 to 10 ml of whole blood from each animal (Montgomery and Sise, 1990). Sequencing of all subclones and PCR products was carried out by the commercial service operated by the University of Otago Centre for Gene Research (ABI 373 automated sequencer).

DNA Markers

Microsatellite (dinucleotide repeat) markers which amplified DNA from sheep were developed within the AgResearch Molecular Biology Unit as previously described (Lord et al 1998) or were from the cattle and sheep mapping literature. Known genes from human chromosome 4 were also analysed for linkage to FecB and placed on the linkage map.

Haplotype Analysis

Markers flanking the critical region for the FecB locus were screened in all daughters from the half-sib families. Individuals with a genetic recombination in a 20 cM region around the FecB locus were identified for subsequent analysis. Additional markers identified within the critical region were typed in the families and/or in the recombinant panel. The critical region was further defined by linkage and haplotype analysis to lie between PDHA2 and JP27 (Table 2).

PCR Amplification of BMP1B Receptor Gene Products

Standard conditions for Polymerase Chain Reaction (PCR) amplification of genomic DNA were used. PCR products containing the single nucleotide mutations were amplified using primers

5′AGTGTTCTTCACCACAGAG; and

5′CATGCCTCATCAACACCG.

The PCR products were identified by electrophoretic separation in 2.5% agarose gels alongside commercially available DNA size markers, extracted from the gels using Qiagen Gel Extraction kit and sent for commercial sequencing.

Forced RFLP

To screen the mutation through the flocks of sheep a method that deliberately introduces a point mutation into one of the. primers was used so that the PCR product will contain an AvaII restriction site. PCR products from non-carrier animals contain no restriction site. Primers 5′ GTCGCTATGGGGAAGTTTGGATG and 5′ CAAGATGTTTTCATGCCTCATCAACACGGTC amplify a 140 bp band, after digestion with AvaII the BB animals will have a 110 bp band, B+ animals will have 140 and 110 bp and the ++ animals will have a 140 bp band. The fragments were amplified using 35 cycles of 94° C. 15 sec, 60° C. 30 sec, 72° C. 30 sec followed by 72° C. 5 min and 99° C. 15 min. The fragments were then electrophoresed on a 2.5% agarose gel and scored for the presence of the mutation.

Reverse Transcriptase-PCR

The expression of the BMPIB receptor in different tissues was determined by PCR from cDNA produced from 0.1 μg of total RNA isolated from tissues from BB ewes or BB rams. Primers 5′AGCTGTGAAAGTGTTCTTCACC and 5′TCTTTTGCTCTGCCCACAAAC amplify across the 1.2 kb intron of BMP1B receptor to produce a 880 bp fragment from cDNA. β-actin primers 5′GCATGGGCCAGAAGGACTCC and 5′ CGTAGATGGGCACCGTGTGG were used as a control.

Results

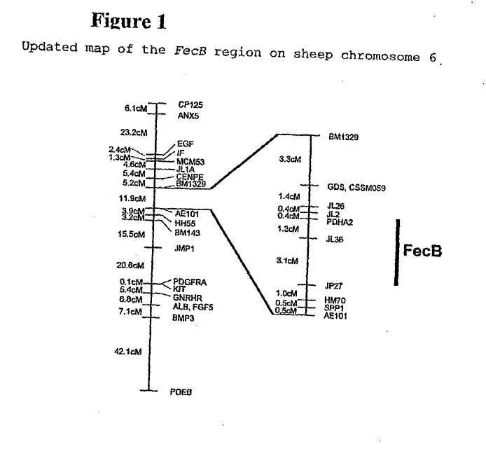

Human BMP1B receptor is found on chromosome 4 at position 4q23-q24. In sheep the FecB gene maps to chromosome 6 (FIG. 1) between markers JL2 and JP27 and we believe it is located very close to JP36. This is based on haplotype analysis of animals that have undergone recombination between known markers (Table 2 and the QTL graph FIG. 1B); and their Booroola phenotype of increased ovulation has been lost or retained.

The entire BMP1B receptor gene from the Booroola and wild-type sheep has been sequenced from amplified PCR products from cDNA isolated from ovary tissue. We found a single nucleotide polymorphism in which an A in the wildtype sheep has been replaced by, a G in the Booroola (FIG. 2). This changes the protein sequence from a glutamine (Q) to an arginine (R) in the Booroola animals (FIGS. 3a-b and FIG. 3c). This represents a change from a neutral amino acid to a basic amino acid. The location of this mutation is within the intracellular signalling domain of the BMP1B receptor.

This single base change has been verified by PCR and sequence analysis of genomic DNA from sheep carrying the Booroola phenotype (Table 1). The mutation we found in BMP1B receptor has not been seen in wildtype animals. We sampled animals from our own stocks of BB, B+ and ++ animals and we also analysed eighty animals that had been sent to Genomnz (diagnostic commercial unit within AgResearch) from the Saudi Arabia, Netherlands and the USA which had been scored by their commercial test and our results were consistent. We found the mutation segregating in their flocks which had derived from the Booroola rams used in their breeding programs.

To date we have screened 300 animals from our backcross and half-sib flocks and found the mutation was consistent by correlating with the phenotype of increased ovulation. We also looked for the mutation in non-Booroola merinos but it was not present. We also examined 65 animals from six different sheep breeds (Coopworths, Perindale, Romney, Texel, Finn, and Gotland) but the mutation was never found in these animals. Hence the mutation we describe has only been found to date in animals derived from the Booroola merino.

TABLE 1
DNA from Animals of known carrier status was amplified by PCR and
the product sequenced. For the heterozygous B+ animals, both the
G and A nucleotides are found in the same peak representing both alleles.

| Animal ID | Genotype | Sequence |
|---|---|---|
| OA771 | BB | CGG |
| OA1012 | ++ | CAG |
| OA1692 | B+ | CG/AG |
| OA1482 | B+ | CG/AG |
| OA2850 | ++ | CAG |
| OA1331 | BB | CGG |
| OA1035 | ++ | CAG |
| OA1684 | B+ | CG/AG |
| OA3725 | ++ | CAG |
| OA8015 | BB | CGG |
| OA8017 | BB | CGG |
| OA5020 | ++ | CAG |
| OA5026 | ++ | CAG |
| 93-W7798-TAM | BB | CGG |
| 97-B9938-AKH | ++ | CAG |
| 97-B9341-AKH | B+ | CG/AG |
| 95-W3232-AKH | ++ | CAG |
| 97-B9453-AKH | B+ | CG/AG |
| 97-2607-TEX | B+ | CG/AG |
| 97-2506-TEX | ++ | CAG |
| 97-2609-TEX | ++ | CAG |
| 97-2553-TEX | B+ | CG/AG |
| 99-990105-IDL | BB | CGG |
| 99-990106-IDL | BB | CGG |
| 98-980315-IDL | B+ | CG/AG |
| 98-980312-IDL | B+ | CG/AG |
| 98-980272-IDL | ++ | CAG |
| 98-980228-IDL | ++ | CAG |

The mutation which we found in the BMP1B receptor has not been seen in wildtype animals. We tested animals from our own stocks of BB, B+ and ++ animals, as well as animals from Saudi Arabia, Netherlands and the USA which had been scored by the Genomnz commercial test, and obtained results consistent with those obtained using the commercial test.

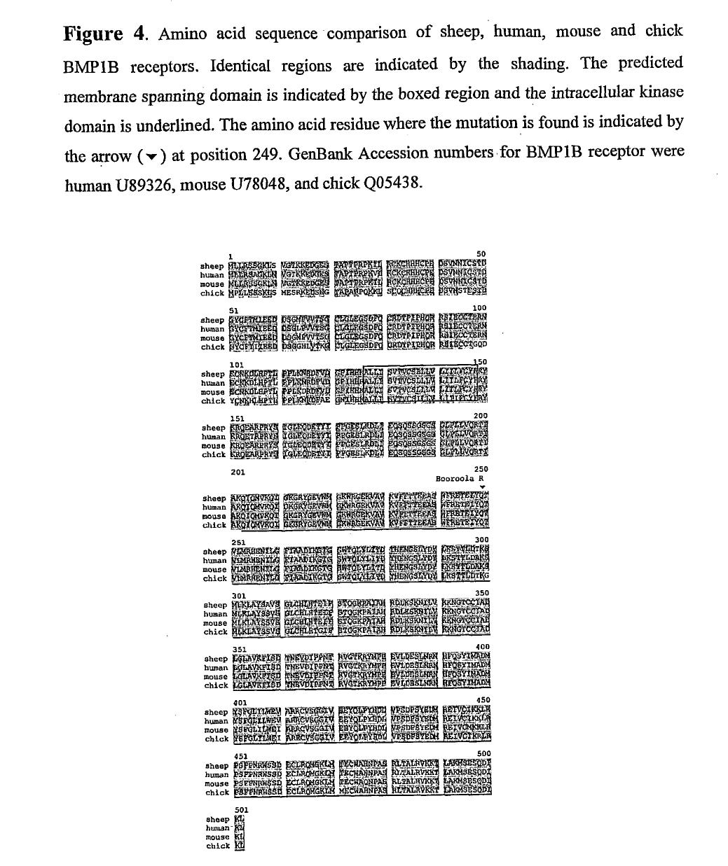

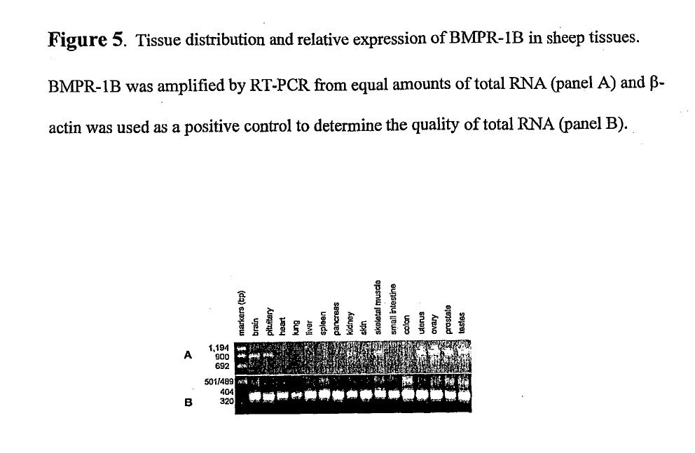

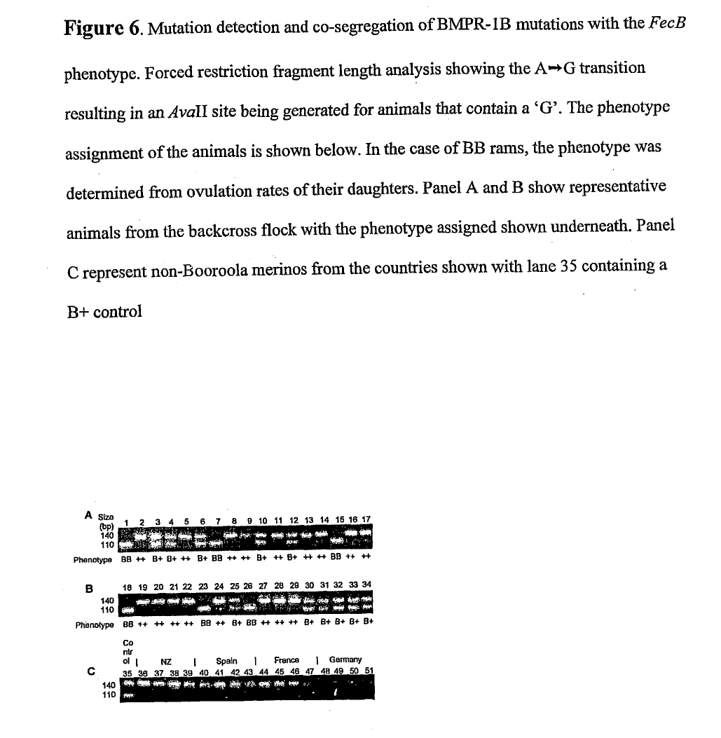

The protein encoded by the BMP1B receptor gene is highly homologous to the human and mouse sequences (FIG. 4), with only two amino acid differences between human and sheep, at positions 298 and 308. The sequence surrounding the critical amino acid 249 is identical in humans and wildtype sheep. It will therefore be appreciated that the modulation of the activity of this gene has potential for use in in vitro fertilization programs, as well as in animal breeding.

Throughout this specification use of the term “comprises” or its grammatical variants is not intended to be limiting. Therefore this term should not be understood as excluding the presence of other features or elements to the present invention. Thus, the word “comprises” as used herein is equivalent to the word “includes”.

Aspects of the present invention have been described by way of example only and it should be appreciated that modifications and additions may be made thereto without departing from the scope thereof as defined in the appended claims.

TABLE 2
Booroola haplotype analysis.

| | Markers | | | | | | | | | | | | |
|---|---|---|---|---|---|---|---|---|---|---|---|---|---|
| Animal | BM1329 | GDS | CSSMO59 | JL26 | JL2 | PDHA2 | JL36 | Phenotype | JP27 | HM70 | AE101 | HH55 | BM143 |
| 880257 | − | − | − | □ | □ | − | □ | + | ● | ● | − | ● | − |
| 870019 | □ | − | □ | □ | □ | − | □ | + | ● | ● | − | − | − |
| 890156 | □ | □ | − | □ | □ | − | □ | + | ● | ● | − | − | ● |
| 911023 | □ | − | − | □ | □ | □ | □ | + | − | ● | ● | − | − |
| 850096 | ● | − | − | ● | ● | − | ● | B | □ | □ | − | □ | □ |
| 900007 | − | − | ● | ● | ● | − | ● | B | □ | □ | − | □ | □ |
| 890037 | ● | − | − | ● | ● | − | ● | B | □ | − | □ | − | □ |
| 900028 | ● | − | ● | ● | ● | − | ● | B | □ | □ | − | □ | □ |
| 890124 | ● | − | − | ● | ● | ● | − | B | □ | □ | − | − | □ |
| 890173 | ● | − | ● | ● | ● | ● | ● | B | − | □ | □ | □ | □ |
| 900085 | − | − | ● | ● | ● | − | − | B | □ | □ | − | − | − |
| 890011 | − | − | □ | − | □ | − | ● | B | ● | ● | − | − | ● |
| 880261 | − | − | □ | □ | □ | − | ● | B | ● | ● | − | ● | − |
| 850055 | □ | □ | □ | □ | □ | □ | ● | B | ● | ● | − | ● | ● |
| 880086 | − | − | □ | □ | □ | □ | ● | B | ● | ● | ● | − | ● |
| 850087 | − | □ | □ | □ | □ | □ | − | + | ● | ● | − | ● | ● |
| 911003 | ● | − | − | ● | ● | − | − | + | □ | − | □ | □ | □ |
| 880457 | − | ● | ● | ● | ● | − | − | + | □ | − | □ | − | □ |

The recombinant animals listed were analysed for inheritance of alleles from either the Booroola ●or non-Booroola □ chromosome, − indicates where the markers were not informative for these animals. The breakpoint is indicated by the column for phenotype where the carrier (B) or non-carrier (+) status of FecB for each animals is shown

References

Crawford A M, Dodds K G, Ede A J, Pierson C A, Montgomery G W, Garmonsway H G, Beattie A E, Davies K, Maddox J F, Kappes S W, Stone R T, Nguyen T C, Penty J M, Lord E A, Broom J E, Buitkamp J, Schwaiger W, Epplen J T, Matthew P, Matthews M E, Hulme D J, Beh K J, McCraw R A, Beattie C W. An autosomal genetic linkage map of the sheep genome. Genetics 1995; 140:703-724.

Davis G H, Montgomery G W, Allison A J, Kelly R W and Bray A R (1982). Segregation of a major gene influencing fecundity in progeny of Booroola sheep. New Zealand Journal Agricultural Research 25:525-529

Fry R C, Clarke I J, Cummins J T, Bindon B M, Piper L R and Cahill L P (1988) Induction of ovulation in chronically hypophysectomized Booroola ewes. Journal of Reproduction and Fertility 82:711-715.

Heldin, C-H, Miyazona K, ten Dijke P (1997) TGF-β signalling from cell membrane to nucleus through SMAD proteins. Nature 390:465-471.

Hogan et al (1966) In “Manipulating the Mouse Embryo”, Cold Spring Habor Lab. Press.

Hudson N L, O'Connell A R, Shaw L, Clarke I J and McNatty K P. (1999) Effect of exogenous FSH on ovulation rate in homozygous carriers or noncarriers of the Booroola FecB gene after hypothalamic-pituitary disconnection or after treatment with a GnRH agonist. Domestic Animal Endocrinology 16:69-80.

Hussainus et al 1999 Stable antisense RNA expression neutralises the activity of low-density lipoprotein receptor-related protein and promotes urokinase accumulation in the medium of an astrocytic tumor cell line. Antisense Nucleic Acid Drug Development volume 9:183-190.

Kretzschmar M, Liu F, Hata A, Doody J and Masague J (1997) The TGF-β family mediator Smad1 is phosphorylated directly and activated functionally by the BMP receptor kinase. Genes and Development 11:984-995.

Lord E A, Davis G H, Dodds K G, Henry H M, Lumsden J M, Montgomery G W. (1998) Proceedings of the 6^{th} World Congress on Genetics Applied to Livestock Production. 27:19-22.

Massague J (1998) TGF-β signal transduction. Annual Review Biochemistry 67:753-791.

McNatty K P, Lun S, Heath D A, Ball K, Smith P, Hudson N L, McDiarmid J, Gibb M and Henderson K M (1986) Differences in ovarian activity between Booroola x Merino ewes which were homozygous, heterozygous and non-carriers of a major gene influencing their ovulation rate. Journal of Reproduction and Fertility. 77:193-205.

McNatty K P, Hudson N, Henderson K M, Gibb M, Morrisson L, Ball K and Smith P. (1987) Differences in gonadotrophin concentrations and pituitary responsiveness to GnRH between Booroola ewes which were homozygous (FF), heterozygous (F+) and non-carriers (++) of a major gene influencing their ovulation rate. Journal of Reproduction and Fertility 80:577-588.

MeNatty K P, Hudon N L, Lun S, Heath D A, Shaw L, Condell L, Phillips D J and Clarke I J (1993) Gonadotrophin-releasing hormone and the control of ovulation rate by the FecB^{B} gene in Booroola ewes. Journal of Reproduction and Fertility 98:97-105.

Montgomery G W and Sise J A (1990) Extraction of DNA from sheep white blood cells. New Zealand Journal of Agricultural Research 33: 437-441.

Montgomery G W, Lord E A, Penty J M, Dodds K G, Broad T E, Cambridge L, Sunden S L F, Stone R T, Crawford A M (1994) The Booroola fecundity (FecB) gene maps to sheep chromosome 6. Genomics 22:148-153.

Onichtchouck et al 1999 Silencing of TGF-beta signalling by the pseudoreceptor BAMBI Nature 401:480-485.

Shimasaki S, Zachow R J, L i D, Kim H, Iemura S-I, Ueno N. Sampath K, Chang R J, Erickson G F (1999) A functional bone morphogenetic protein system in the ovary. Proceedings National Academy Science 96:7282-7287.

1. An isolated mutated nucleic acid molecule encoding the BMP1B receptor polypeptide wherein the molecule:
(a) has a sequence which differs from that of the wild type BMP1B receptor polypeptide in that the codon encoding amizno-acid residue 249 encodes arginine rather than glutamine;
(b) is a non-wildtype variant of the sequence defined in (a) having an affect on modulation of ovulation;
(c) is the complement of the molecule defined in (a) or (b)y or
(d) is an anti-sense sequence corresponding to any of the sequences in (a)-(c);

2. An oligonucleotide probe capable of hybridizing under stringent conditions to a nucleic acid molecule according to claim 1, in which the probe comprises:
(a) the codon encoding amino-acid residue 249 of die mutated BMP1B receptor, or (b) bar a sequence complementary to (a).

3. An isolated nucleic acid Molecule as claimed in claim 1 wherein the nucleotide sequence of the molecule in (a) of claim 1 is set forth in SEQ ID No. 3.

4. A method for identifying a vertebrate which carries a mutated BMP1B receptor nucleic acid molecule, said method comprising the steps of:
i) obtaining a tissue or blood sample from the vertebrate;
ii) isolating DNA from the sample;
iii) optionally isolating BMP1B receptor DNA from DNA obtained at step ii);
iv) optionally probing said DNA with a probe complementary to the mutated BMP1B receptor molecule of claim 1, thereby to identify mutated BMP1B receptor;
v) optionally amplifying the amount of mutated BMP1B receptor DNA and;
viii) determining whether the mammal BMP1B receptor sequence DNA obtained in step (ii) carries a mutation which is associated with increased or decreased ovulation rates.

5. A method according to claim 3, in which the vertebrate is male or female, and carries a single copy of the mutated BMP1B receptor nucleic acid molecule.

6. A method according to claim 4, in which the vertebrate is female, and carries two copies of the mutated BMP1B receptor nucleic acid molecule.

7. A method as claimed in any one of claims 4 to 6 wherein the vertebrate is selected from the group consisting of humans, sheep, cattle, horses, goats, deer, poultry, pigs, cats, dogs, and possums.

8. A genetic marker for increased ovulation rate in a vertebrate, comprising a nucleic acid molecule which specifically hybridises to the nucleotide sequence of claim 1, or to a variant or complement thereof.

9. A genetic marker as claimed in claim 8 which comprises a fragment of a mutated nucleotide sequence in the region which includes the codon encoding amino acid residue 249.

10. A genetic marker as claimed in claim 8 or claim 9 in which the vertebrate is selected from the group consisting of humans, sheep, goats, cattle, horses, deer, pigs, poultry, cats, dogs, and possums.

11. An isolated BMP1B receptor polypeptide, having an amino acid sequence which differs from the wild type in that residue 249 is arginine not glutamine.

12. An isolated BMP1B receptor polypeptide as claimed in claim 11 wherein the amino acid sequence of the polypeptide is set forth in SEQ ID No. 4.

13. An isolated BMP1B receptor polypeptide having an amino acid sequence in which residue 249 is glutamine, but which is otherwise different from the wildtype BMP1B polypeptide sequence and which has the ability to modulate ovulation in a female mammal.

14. The use of SEQ ID No. 2 in the modulation of ovulation in a vertebrate.

15. An isolated nucleic acid molecule encoding the polypeptide of any one of claims 11 to 13.

16. A vector comprising the nucleic acid molecule of claim 1 or claim 15.

17. A host cell which has been transformed by a vector as claimed in claim 16.

18. A method of modulating the ovulation rate of a female vertebrate comprising the step of administering to said vertebrate an effective amount of mutated or wild type BMP1B receptor.

19. A method of increasing the ovulation rate of a female vertebrate, comprising the step of administering to said vertebrate an effective amount of a polypeptide according to claims 11 or 12 or a polypeptide according to claim 13 which have the ability to increase the ovulation rate of a female vertebrate.

20. A method of reducing the ovulation of a female vertebrate comprising the step of administering an effective amount of an agent selected from the group consisting of:
a) an immunising-effective amount of wild-type or mutated BMP1B receptor polypeptide, or an immunogenic region thereof;
b) an antibody directed against wild-type or mutated BMP1B receptor polypeptide, or an antigen-binding fragment thereof;
c) an antisense nucleic acid directed against nucleic acid encoding the mutated or wild-type BMP 1B receptor polypeptide;
d) a pseudoreceptor to the wild-type or mutated BMP1B receptor; and
e) a ligand which binds to the wild-type or mutated BMP1B receptor polypeptide, to thereby inhibit the activity of the endogenous BMP1B receptor of the vertebrate.

21. A method according to claim 20, in which the agent is an antibody as defined in claim 20(b).

22. A method according to claim 21, in which the antibody is a monoclonal antibody.,

23. A method according to claim 20, in which the agent is an antisense nucleic acid.

24. A method according to claim 23, in which the antisense nucleic acid is directed to a nucleic acid encoding the BMP1B receptor polypeptide of any one of claims 11 to 14.

25. A method according to claim 20, in which the agent is a pseudoresceptor.

26. A method according to claim 25, in which the pseudoreceptor is directed against the receptor according to any one of claims 11 to 14.

27. A method according to claim 20, in which the agent is a ligand.

28. A method according to claim 27, in which the ligand binds to the BMP1B receptor polypeptide of any one of claims 11 to 14.

29. A composition comprising a polypeptide according to any one of claims 11 to 13, and a pharmaceutically or veterinarily acceptable carrier.

30. A composition comprising a nucleic acid molecule according to claim 1, or a nucleic acid molecule encoding a polypeptide according to any one of claims 11 to 13, and a pharmaceutically or veterinarily acceptable carrier.

31. The use of a composition comprising an effective amount of agent selected from the group consisting of:
(a) wild-type or mutated BMP1B receptor polypeptide, or an immunogenic region thereof:
(b) an antibody directed against wild-type or mutated BMP1B receptor polypeptide, or an antigen-binding fragment thereof;
(c) an antisense nucleic acid directed against nucleic acid encoding the mutated or wild-type BMP1B receptor polypeptide;
(d) a pseudoreceptor to the wild-type or mutated BMP1B receptor; and
(c) a ligand which binds to the wild-type or mutated BMP1B receptor polypeptide, to thereby inhibit the activity of the endogenous BMP1B receptor of the vertebrate;
and a pharmaceutically or veterinarily acceptable carrier, to modulate the ovulation rate of a vertebrate.

32. A kit for identifying vertebrates which carry a mutated BMP1B receptor, said kit comprising:
a) primer pairs for amplification of the appropriate region of the BMP1B receptor gene and optionally one or more of;
b) buffer solution for the DNA amplification;
c) a mixture of deoxynucleotides;
d) means for DNA amplification;
e) control DNA from the species being tested;
f) appropriate standards; and
g) a detection system.

33. A kit according to claim 32, in which the means for DNA amplification is a thermostable polymerase enzyme, and the amplification is performed by polymerase chain reaction.

34. A kit for detecting circulating mutated BMP1B receptor polypeptide in a vertebrate, wherein said kit comprises an antibody directed to the mutated polypeptide.

35. A kit according to claim 34, in which the anti-body is a monoclonal antibody.

36. The use of a nucleic acid molecule able to hybridise under stringent conditions to the molecule of claim 1 to modulate the ovulation rate of a vertebrate.

37. An isolated mutated nucleic acid molecule substantially as described herein with reference to any example, drawing or sequence listing thereof, relating to non wildtype nucleic acid molecules.

38. A method for identifying a vertebrate which carries a mutated BMP1B receptor nucleic acid molecule, substantially as described herein, with reference to any example and/or drawing thereof.

39. A genetic marker substantially as described herein with reference to any example. or drawing thereof.

40. An isolated mutated BMP1B receptor polypeptide substantially as described herein with reference to any example, drawing or sequence listing thereof.

41. A method of modulating the ovulation rate of a female vertebrate substantially as described herein with reference to any example and/or drawing thereof,

42. A composition comprising a mutated polypeptide substantially as described herein with reference to any example and/or drawing thereof.

43. A composition comprising a nucleic acid molecule substantially as described herein with reference to any example and/or drawing thereof, relating to non wildtype nucleic acid molecules.

44. A kit for identifying vertebrates carrying mutated BMP1B receptor substantially as described herein with reference to any example and/or drawing thereof.


SEQUENCE LISTING (12 sequences)

SEQ ID NO: 1 - DNA, 1612 bp, Ovis aries
  CDS (85)...(1593)
ttttccgttgagctatgacaagagaggatacaaaaagttaaacaagcaagcctgtcatacgtagaagcaaacttccttgataacatgcttttgcgaagttcaggaaaattaagtgtgggcaccaagaaagaggatggtgagagtacagcccccacccctcgtccaaagatcttgcgatgtaaatgccaccaccattgtccagaagactcggtcaacaatatttgcagcacagatggatattgtttcacgatgatagaagaagatgactctgggatgcctgtggtcacttctggatgtctaggactagaaggctcagattttcagtgtcgggacactcccattcctcatcagagaagatccattgaatgctgcacagaacggaatgaatgtaataaagatctgcaccccacacttcctccactgaaaaacagagattttgttgacggacctatacaccacaaagctttacttatatctgtgactgtgtgtagtttgctcttggtcctcatcattttattctgttacttcaggtataaaagacaagaagccagacctcggtacagcattgggttagaacaggacgaaacttacattcctcctggagaatccctgagagacttaattgagcagtcgcagagctcagggagcggatcaggcctccctctgctggtccagaggacaatagcaaagcaaattcagatggtgaaacagattggaaaaggtcgctatggggaagtttggatgggaaagtggcgtggcgaaaaggtagctgtgaaagtgttcttcactacagaggaggccagctggttccgagagacagaaatatatcagacggtgttgatgaggcatgaaaacatcttgggcttcattgctgcagatatcaaagggacggggtcctggacacaactgtacctaatcacagattatcatgaaaatggttccctctatgattacctgaagtccaccaccctagacactaagtcgatgttgaagctagcctattccgcagtcagtggcctctgtcacttacacactgaaatctttagcactcaaggcaaaccagcaattgcccatcgagatctgaaaagtaagaacatcctggtgaagaaaaatggaacttgctgtatagctgacctgggcttggctgttaagtttattagtgacacgaatgaagttgacataccacccaacactcgagttggcaccaagcgctacatgcctccagaagtgttggatgagagcttgaacagaaatcactttcagtcttacatcatggccgacatgtacagttttggactcatcctttgggaggtcgctaggagatgtgtgtcaggaggtatagtggaagaatatcagctcccctatcatgacctggtgcccagtgacccctcttacgaggacatgagagagatcgtgtgtatcaagaagctgcggccctccttccccaaccggtggagcagtgacgagtgtctcaggcagatggggaaactcatgacggaatgctgggctcacaatcctgcctcaagactgacagccctacgggttaagaaaacccttgccaaaatgtcagagtcccaggacattaagctctgaggcaagagtaagtgtctct

SEQ ID NO: 2 - protein, 502 aa, Ovis aries
MLLRSSGKLSVGTKKEDGESTAPTPRPKILRCKCHHHCPEDSVNNICSTDGYCFTMIEEDDSGMPVVTSGCLGLEGSDFQCRDTPIPHQRRSIECCTERNECNKDLHPTLPPLKNRDFVDGPIHHKALLISVTVCSLLLVLIILFCYFRYKRQEARPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDTKSMLKLAYSAVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 3 - DNA, 1612 bp, Ovis aries
ttttccgttgagctatgacaagagaggatacaaaaagttaaacaagcaagcctgtcatacgtagaagcaaacttccttgataacatgcttttgcgaagttcaggaaaattaagtgtgggcaccaagaaagaggatggtgagagtacagcccccacccctcgtccaaagatcttgcgatgtaaatgccaccaccattgtccagaagactcggtcaacaatatttgcagcacagatggatattgtttcacgatgatagaagaagatgactctgggatgcctgtggtcacttctggatgtctaggactagaaggctcagattttcagtgtcgggacactcccattcctcatcagagaagatccattgaatgctgcacagaacggaatgaatgtaataaagatctgcaccccacacttcctccactgaaaaacagagattttgttgacggacctatacaccacaaagctttacttatatctgtgactgtgtgtagtttgctcttggtcctcatcattttattctgttacttcaggtataaaagacaagaagccagacctcggtacagcattgggttagaacaggacgaaacttacattcctcctggagaatccctgagagacttaattgagcagtcgcagagctcagggagcggatcaggcctccctctgctggtccagaggacaatagcaaagcaaattcagatggtgaaacagattggaaaaggtcgctatggggaagtttggatgggaaagtggcgtggcgaaaaggtagctgtgaaagtgttcttcactacagaggaggccagctggttccgagagacagaaatatatcggacggtgttgatgaggcatgaaaacatcttgggcttcattgctgcagatatcaaagggacggggtcctggacacaactgtacctaatcacagattatcatgaaaatggttccctctatgattacctgaagtccaccaccctagacactaagtcgatgttgaagctagcctattccgcagtcagtggcctctgtcacttacacactgaaatctttagcactcaaggcaaaccagcaattgcccatcgagatctgaaaagtaagaacatcctggtgaagaaaaatggaacttgctgtatagctgacctgggcttggctgttaagtttattagtgacacgaatgaagttgacataccacccaacactcgagttggcaccaagcgctacatgcctccagaagtgttggatgagagcttgaacagaaatcactttcagtcttacatcatggccgacatgtacagttttggactcatcctttgggaggtcgctaggagatgtgtgtcaggaggtatagtggaagaatatcagctcccctatcatgacctggtgcccagtgacccctcttacgaggacatgagagagatcgtgtgtatcaagaagctgcggccctccttccccaaccggtggagcagtgacgagtgtctcaggcagatggggaaactcatgacggaatgctgggctcacaatcctgcctcaagactgacagccctacgggttaagaaaacccttgccaaaatgtcagagtcccaggacattaagctctgaggcaagagtaagtgtctct

SEQ ID NO: 4 - protein, 502 aa, Ovis aries
MLLRSSGKLSVGTKKEDGESTAPTPRPKILRCKCHHHCPEDSVNNICSTDGYCFTMIEEDDSGMPVVTSGCLGLEGSDFQCRDTPIPHQRRSIECCTERNECNKDLHPTLPPLKNRDFVDGPIHHKALLISVTVCSLLLVLIILFCYFRYKRQEARPRYSIGLEQDETYIPPGESLRDLIEQSQSSGSGSGLPLLVQRTIAKQIQMVKQIGKGRYGEVWMGKWRGEKVAVKVFFTTEEASWFRETEIYRTVLMRHENILGFIAADIKGTGSWTQLYLITDYHENGSLYDYLKSTTLDTKSMLKLAYSAVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 5 - DNA, 19 bp, Artificial Sequence
  Primer
agtgttcttcaccacagag

SEQ ID NO: 6 - DNA, 18 bp, Artificial Sequence
  Primer
catgcctcatcaacaccg

SEQ ID NO: 7 - DNA, 23 bp, Artificial Sequence
  Primer
gtcgctatggggaagtttggatg

SEQ ID NO: 8 - DNA, 31 bp, Artificial Sequence
  Primer
caagatgttttcatgcctcatcaacacggtc

SEQ ID NO: 9 - DNA, 22 bp, Artificial Sequence
  Primer
agctgtgaaagtgttcttcacc

SEQ ID NO: 10 - DNA, 21 bp, Artificial Sequence
  Primer
tcttttgctctgcccacaaac

SEQ ID NO: 11 - DNA, 20 bp, Artificial Sequence
  Primer
gcatgggccagaaggactcc

SEQ ID NO: 12 - DNA, 20 bp, Artificial Sequence
  Primer
cgtagatgggcaccgtgtgg